(19) United States
(12) Patent Application Publication
(10) Pub. No.: US 2011/0245676 A1
(43) Pub. Date: Oct. 6, 2011

(54) METHOD AND APPARATUS FOR ULTRASOUND SIGNAL ACQUISITION AND PROCESSING

(75) Inventors: Feng Lin, Niskayuna, NY (US); Kenneth Wayne Rigby, Clifton Park, NY (US)

(73) Assignee: General Electronic Company, Schenectady, NY (US)

(21) Appl. No.: 12/751,834

(22) Filed: Mar. 31, 2010

(51) Int. Cl. A61B 8/14

(52) U.S. Cl. 600/447; 600/459

(57) ABSTRACT

The present disclosure relates to the measurement of acoustic noise during ultrasound imaging. In one embodiment, a receive beam is directed in a different direction from a transmit beam when an acoustic noise signal is being measured. When a tissue signal is being measured, the receive beam is directed in substantially the same direction as the transmit beam.

### BACKGROUND OF THE INVENTION

The subject matter disclosed herein relates generally to ultrasound imaging, and more particularly, to processing signals acquired during ultrasound imaging.

Medical diagnostic ultrasound is an imaging modality that employs ultrasound waves to probe the acoustic properties of the body of a patient and produce a corresponding image. Generation of ultrasound beams and detection of the reflected energy is typically accomplished via a plurality of transducers located in a probe that is contacted with the patient. Such transducers typically include electromechanical elements capable of converting electrical energy into mechanical energy for transmission and mechanical energy back into electrical energy for receiving purposes.

However, artifacts may be present in images generated using the reflected ultrasound energy. for example, acoustic noise may be present which is composed of echoes not generated at the focal point for reception of reflected ultrasound energy. Such acoustic noise may degrade the contrast resolution of the images and may lead to smears or smearing within the generated images. Further, such acoustic noise may be difficult to separate from the signal of interest because the acoustic noise is only present when ultrasound energy is transmitted into the tissue. As a result, there is no readily available reference with which to establish the level of acoustic noise that may be generated when an ultrasound beam is directed at a region of tissue.

### BRIEF DESCRIPTION OF THE INVENTION

In one embodiment, of the present disclosure, an ultrasound system is provided. In accordance with this embodiment, the ultrasound system includes a probe comprising a plurality of transducer elements and a station in communication with the probe. The station includes transmit circuitry that controls the emission and direction of a transmitted ultrasound beam by the probe, receive circuitry that controls the reception and direction of a received ultrasound beam by the probe, and a controller directing operation of the transmit circuitry and the receive circuitry. The receive circuitry controls the direction of the received ultrasound beam to be the same as the transmitted beam when a tissue signal is being measured. The receive circuitry controls the direction of the received ultrasound beam to be different than the transmitted beam when an acoustic noise signal is being measured.

In an additional embodiment, a method for measuring acoustic noise associated with an ultrasound signal is provided. In accordance with this method, a transmit beam is generated having a transmit steering direction determined at least in part by a transmit delay profile associated with the transmit beam. A receive beam having a receive steering direction determined at least in part by a receive delay profile is also generated. The receive delay profile associated with the receive beam results in substantially the same receive steering direction as the transmit steering direction when a tissue signal is being acquired. The receive delay profile associated with the receive beam results in a receive steering direction that differs from the transmit steering direction when an acoustic noise signal is being measured.

In a further embodiment, a method for processing an ultrasound image is provided. In accordance with this method, a tissue signal and an acoustic noise signal are acquired. An acoustic noise level is derived from the acoustic noise signal. A signal-to-noise ratio is derived using the tissue signal and the acoustic noise signal. The signal-to-noise ratio is processed to generate a weighting profile. The weighting profile is applied to the tissue signal to generate a noise-compensated image.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts components on an ultrasound imaging system, in accordance with an embodiment of the present disclosure;

FIG. 2 depicts an example of a beam profile in accordance with an embodiment of the present disclosure;

FIG. 3 depicts an example of a transmit beam and a receive beam in accordance with an embodiment of the present disclosure; and

FIG. 4 depicts a flowchart illustrating control logic associated with an algorithm for improving image quality in accordance with an embodiment of the present disclosure.

### DETAILED DESCRIPTION OF THE INVENTION

The present disclosure is directed to the measurement and removal of acoustic noise in ultrasound imaging. In accordance with embodiments of this disclosure, a delay profile for beamforming is implemented that suppresses the tissue signal while still generating acoustic noise. As a result, a measure of acoustic noise with little or no tissue signal is generated. The measure of acoustic noise may serve as an image quality indicator, may be used to improve tissue segmentation, and/or may be used for image quality enhancement.

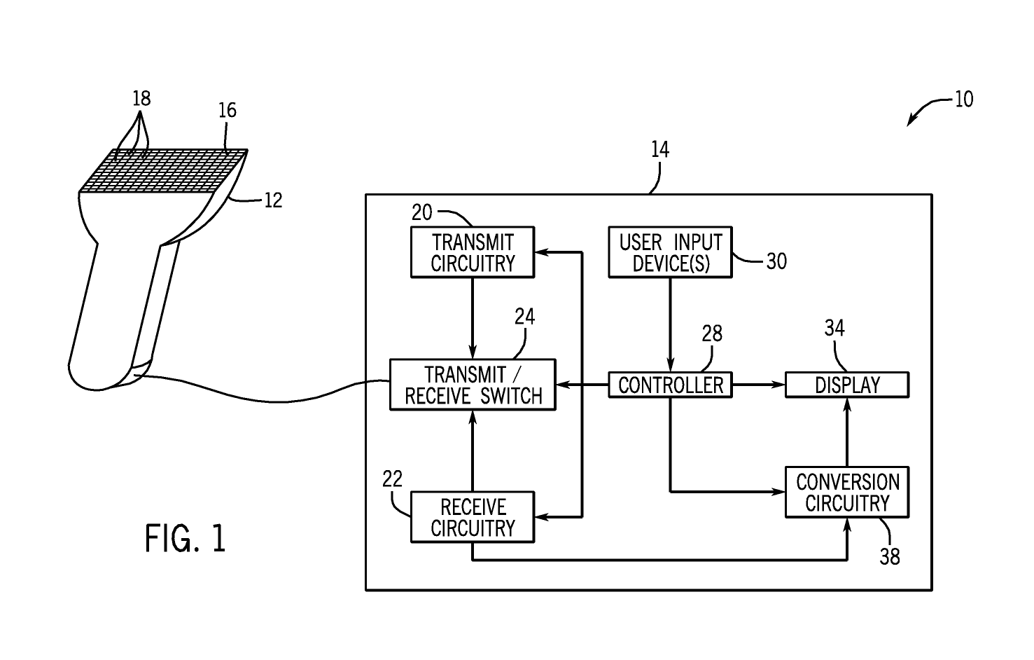

As shown in FIG. 1, an ultrasonic imaging system 10 may include a variety of components, including a handheld probe 12 which is contacted with the patient during an ultrasound examination. In the depicted embodiment, the handheld probe 12 is in communication, such as via a wired or wireless communication link, with an ultrasound system or station 14 which controls operation of the probe 12 and/or processes data acquired via the probe 12.

In one embodiment, the probe 12 includes a patient facing or contacting surface that includes a transducer array 16 having a multitude of transducers 18 that are each capable of producing ultrasonic energy when energized by a pulsed waveform produced by transmit circuitry 20 within the station 14. The ultrasonic energy reflected back toward the transducer array 16, such as from the tissue of a patient, is converted to an electrical signal the transducers 18 of the array 16 and the electrical signal is communicated to receive circuitry 22 of the station 14 for further processing to generate one or more ultrasound images. Operation of the transmit and receive functions of the transducers 18 may be controlled by one or more transmit/receive (T/R) switches 24 within the station 14 that control which of the transmit circuitry 20 or the receive circuitry 22 are in communication with the probe 12 at a given time. As will be appreciated, as used herein the term “circuitry” may describe hardware, software, firmware, or some combination of these which are configured or designed to provide the described functionality, such as transmit beamforming, receive beamforming, and/or scan conversion.

The transmitter circuitry 20, receiver circuitry 22, and/or T/R switches 24 are operated under control of a controller 28 that may operate in response to commands received from a human operator, such as via one or more user input devices 30 (e.g., a keyboard, touchscreen, mouse, buttons, switches, and so forth). In one embodiment, the controller 28 may be implemented as one or more processors, such as general-purpose or application-specific processors, in communication with other respective circuitry and/or components of the station 14.

An ultrasound scan is performed by using the probe 12 and station 14 to acquire a series of echoes generated in response to transmission of ultrasound energy into the tissue of a patient. During such a scan, when the T/R switches 24 are set to transmit, the transmitter circuitry 20 is gated ON momentarily to energize each transducer 18. T/R switches 24 are subsequently set to receive, and the echo signals received by each transducer 18 are communicated to the receive circuitry 22. The separate echo signals from each transducer 18 are combined in the receive circuitry 22 into a signal which is used to produce a line in an image displayed on a display 34 incorporated in or in communication with the station 14.

In one embodiment, the transmit circuitry 20 may be configured to operate the array of transducers 16 such that the ultrasonic energy emitted is directed, or steered, as a beam. For example, the transmit circuitry 20 can impart respective time delays to generate temporally offset pulsed waveforms that are applied to respective transducers 18. These temporal offsets result in differential activation of the respective transducers 18 such that the wavefront of ultrasound energy emitted by the transducer array 16 is effectively steered or directed in different directions with respect to the surface of the transducer array 16. Thus, by adjusting the time delays associated with the pulsed waveforms that energize the respective transducers 18, the ultrasonic beam can be directed toward or away from an axis associated with surface of the transducer array 16 by a specified angle (θ) and focused at a fixed range, R, within the patient tissue. In such an implementation, a sector scan is performed by progressively changing the time delays in successive excitations. The angle θ is thus incrementally changed to steer the transmitted beam in a succession of steering directions.

The echo signals produced by each burst of ultrasonic energy reflect are differentially reflected by structures or structure interfaces located at successive ranges along the ultrasonic beam. The echo signals are sensed separately by each transducer 18 and a sample of the echo signal magnitude at a particular point in time represents the amount of reflection occurring at a specific range. However, due to the differences in the propagation paths between a reflecting structure and each transducer 18, these echo signals may not be detected simultaneously. Therefore, in one embodiment, the receive circuitry 22 amplifies the separate echo signals, imparts the proper time delay to each, and sums them to provide a single echo signal which represents the total ultrasonic energy reflected from a point or structure located at range R along the ultrasonic beam oriented at the angle θ.

To simultaneously sum the electrical signals produced by the echoes detected at each transducer 18, time delays are introduced into the separate channels defined in the receive circuitry 22. In conventional ultrasound scans, the time delays for reception correspond to the time delays associated with transmission, described above, such that the receive beam has a corresponding steering direction as the transmit beam. That is, the steering direction from which ultrasound energy is received generally corresponds to the steering direction in which the ultrasound energy was transmitted. However, the time delay associated with each receive channel may be adjusted or changed during reception of the echo to provide some degree of dynamic focusing of the received beam at the range R from which the echo signal emanates. In embodiments of the present disclosure, as discussed herein, the delay profile employed for reception by the receive circuitry 22 may differ from the corresponding delay profile employed by the transmit circuitry 20 such that the receive circuitry is effectively looking or scanning in a different direction from where the transmitted ultrasound energy is directed, i.e., the steering direction of the receive beam differs from the steering direction of the transmit beam.

For example, during acquisition of image data acquisition, the controller 28 provides the specified delays to the receive circuitry 22 to receive echo data along the direction θ, corresponding to the beam steered by the transmit circuitry 20, and samples the echo signals at a succession of ranges R so as to provide the proper delays and phase shifts to dynamically focus at points P along the beam. Thus, each emission and reception of an ultrasonic pulse waveform during an image acquisition portion of an examination results in acquisition of a series of data points which represent the amount of reflected sound from a corresponding series of points P located along the ultrasonic beam.

In accordance with the present disclosure, acoustic noise data is also acquired during an examination. During acquisition of the acoustic noise signal, the controller 28 provides a different set of delays to the receive circuitry 22 to receive echo data from a direction other than θ, such that echo data is received from directions other than direction of the transmitted ultrasound beam. Thus, each emission and reception of an ultrasonic pulse waveform during an acoustic noise measuring portion of an examination results in acquisition of a series of data points which represent the amount of reflected sound from directions other than that in which the ultrasound beam is directed.

Conversion circuitry 38 receives the various series of data points produced by the receive circuitry 22 and converts the data into the desired image and/or noise measurements. Alternatively, the controller 28 and/or other processor-based components of the station 14 may process the signals generated by the receive circuitry 22 that correspond to acoustic noise to generate measurements or other characterizations of the acoustic noise for display or for use by the conversion circuitry 38 in generating images.

In one embodiment, the conversion circuitry 38 converts the acoustic image data from polar coordinate (R-θ) sector format or Cartesian coordinate linear array to appropriately scaled Cartesian coordinate display pixel data suitable for display at a specified frame rate. This scan-converted acoustic data is then supplied to the display 34, which, in one embodiment, images the time-varying amplitude of the signal envelope as a grey scale.

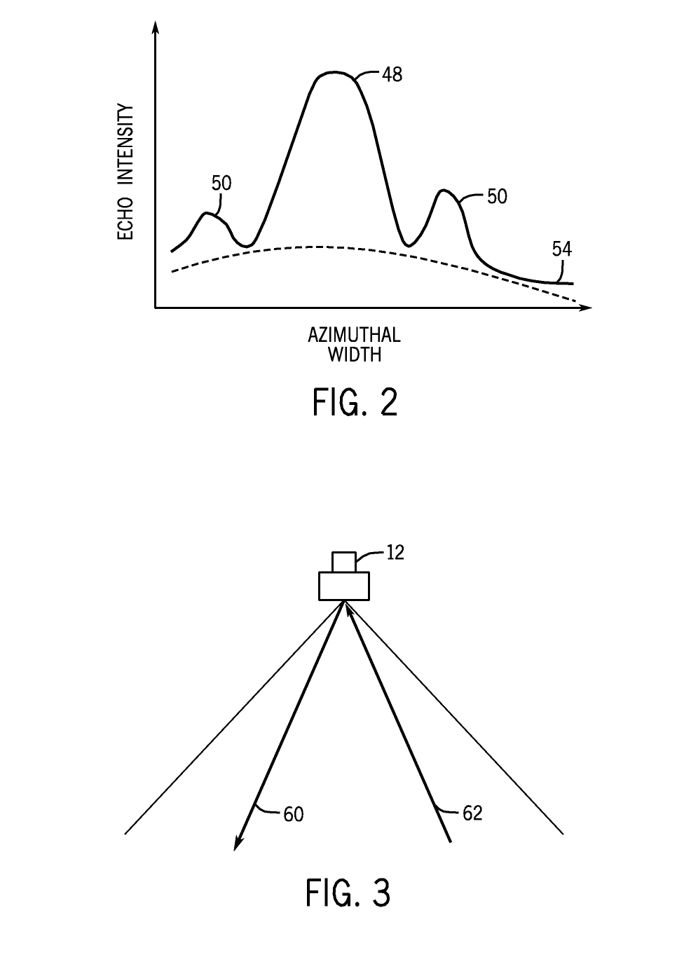

With the foregoing system discussion in mind, the present disclosure is related to various approaches by which acoustic noise may be ascertained and used in ultrasound image acquisition and processing. As discussed herein, acoustic noise in the ultrasound context relates to signal (e.g., echoes) not originating from the focus to be imaged. For example, turning to FIG. 2 where main lobe 48 corresponds to tissue signal at the focus, acoustic noise may occur due to the presence of side lobes 50 (shown with respect to acoustic noise floor 54 and main lobe 48), grating lobes, out-of-plane signals, near-field reverberations from tissue or bones, and defocusing caused by phase aberration. The presence of acoustic noise may cause artifacts (such as smearing and/or degraded contrast resolution) in the reconstructed image or images. Unlike electronic noise, which may be measured by turning off the transmit signal, acoustic noise is only present when the transmit signal is on, making acoustic noise difficult to separate from the tissue signal.

In accordance with the present disclosure, general and specific frameworks are described for measuring acoustic noise. In particular, approaches are discussed for suppressing the tissue portion of the measured signal while keeping the acoustic noise portion of the signal. In accordance with one embodiment, this is implemented by manipulating the delay profile associated with beamforming to cancel out coherent signals in the receive beam while keeping background noise. That is, the delay profile of the receive beam is manipulated to steer the receive beam away from the transmit beam. Such delay profile manipulations may be performed in accordance with:

$$b ( t ) = {\sum }_{i} {a}_{i} {c}_{i} ( t + {d}_{i} ) \qquad ( 1 )$$

where b(t) is the beamformed signal, i is the channel index, aᵢ is the apodization weighting, cᵢ(t) is the channel signal, and dᵢ is the channel delay. In accordance with one embodiment, the channel delay d is manipulated such that the receive beam is derived for various directions, e.g., directions other than that associated with the transmit beam. That is, when measuring acoustic noise, the direction of the receive beam is not the same as the direction of the transmit beam. In this manner, the delay for each channel may be specified to minimize or reduce the tissue signal at the focus while maximizing or increasing acoustic noise.

In a more general format, the acoustic noise can be evaluated using a combination of multiple beamformed signals each having different delay configuration. For example, such an approach may be performed in accordance with:

$$s ( t ) = {\sum }_{m} {w}_{m} {b}_{m} ( t ) \qquad ( 2 )$$

where s(t) is the estimated acoustic noise, m is the index of a beamforming configuration, wₘ is a weighting factor, and bₘ(t) is the beamformed signal for each configuration. In accordance with this approach, it is possible to obtain a general knowledge of the noise floor by looking in multiple direction.

In certain embodiments, the different transmit and receive profiles may be implemented by using suitable computer code, e.g., software or firmware, implemented on the system 10 or by using general purpose or application specific circuitry provided in the system 10. In one such embodiment, the beamforming module can be a conventional beamforming module, which is typically implemented and hardware and, therefore, may be difficult to change. Thus, in this manner, such difference in transmit and receive delay profiles may be implemented in conventional ultrasound imaging systems without changes to the hardware and/or without otherwise modifying the components of the system. Instead, such an approach may be implemented via manipulation of the control signals (e.g., delays, pulse waveforms, and so forth) sent to the probe 12. Such control signal manipulations may be implemented in software, firmware, and/or hardware associated with the system 10. For example, in certain embodiments, beamforming may be implemented in software on the system 10, allowing the respective different delay profiles for transmit and receive beamforming to be implemented in software, without change or modification to the existing hardware of the system 10.

With the foregoing in mind, in one embodiment the transmit and receive delay profiles are set so that the transmitting beam 60 and the receiving beam 62 are focused in different directions, as depicted in FIG. 3. For example, the angular offset between the transmitting beam 60 and the receiving beam 62 may, in one embodiment, be between 15° to 60° (e.g., 45°, 50°, and so forth). In this manner, the receiving beam 62 may be directed away from the main lobe 48 generated by a transmit beam 60 (FIG. 2) and may instead contain signals from the side lobes 50 or from other signal components contributing to acoustic noise. In this manner, the average or median level of acoustic noise may be estimated.

For example, in one embodiment the whole beam profile may be evaluated for all of the transmitting beams that form an image plane or image volume. In one implementation this is accomplished by collecting the beamformed signals at multiple angles for each transmitting beam. Such a beam profile may be useful in discerning where there are significant side lobes and/or acoustic noise. Further, the beam profile may be a good indicator of image quality since side lobes and/or acoustic noise are typically associated with poor image quality and/or degraded images.

However, as will be appreciated, the time or data volume involved in measuring the entire beam profile for all of the transmitting beams that form an image plane or image volume may be prohibitive. Therefore, in alternative embodiments, for each transmitting beam signals may be measured at only one or a few (e.g., two, three, four, five, eight, ten, and so forth) representative angles offset from the angle of the transmit beam. A measure of the acoustic noise (e.g., the average acoustic noise) may then be derived based on these limited samples.

To implement the acoustic noise measurement during an examination or scan protocol several approaches may be employed. In one approach, measurement of acoustic noise may be interleaved with the acquisition of tissue signals. For example, a frame or several beams of acoustic noise data (i.e., where the receive beam angle does not equal the transmit beam angle) may be alternatingly obtained between acquisitions of tissue signal data (i.e., where the receive beam angle equals the transmit beam angle). For example, in such an interleaved embodiment, one frame of acoustic noise data may be obtained for every frame of tissue signal data (i.e. 1:1) or one frame of acoustic noise data may be obtained for some larger number of frames of tissue signal data (i.e., 1:2. 1:3. 1:4, 1:5, and so forth).

Alternatively, acoustic noise and tissue signal data may be acquired simultaneously. For example, in implementations where the ultrasound system 10 has spare or unused multiple line acquisition capabilities, this unused multiple line acquisition capability maybe used to simultaneously acquire signals related to acoustic noise, as discussed above, in conjunction with the tissue signal data.

As will be appreciated, the acoustic noise measurement as discussed herein may have a variety of applications. For example, measures of acoustic noise may serve as an indication of image quality with respect to contrast resolution, which may in turn provide indications of proper probe contact and/or the degree of phase aberration. For example, if the probe does not have a good contact with the tissue and a portion of the transducer elements is not able to collect signal from tissue, the main lobe of the beam profile, as shown in FIG. 2, is spread out and the shape of the beam profile indicates which portion of the transducer elements is not functioning. As another example, when severe aberration is present in the sound path, the ultrasound wavefront may be distorted and the side lobes of the beam profile raised significantly. The side lobe level difference from an ideal case without aberration indicates the degree of the aberration. Further, measures of acoustic noise may be used as a reference for adaptive or dynamic optimization and/or for image quality enhancement. In addition, to the extent that image contrast may be improved or enhanced using measures of acoustic noise, such improved images may allow better segmentation and/or separation, either visually or by application of computer-implemented segmentation algorithms.

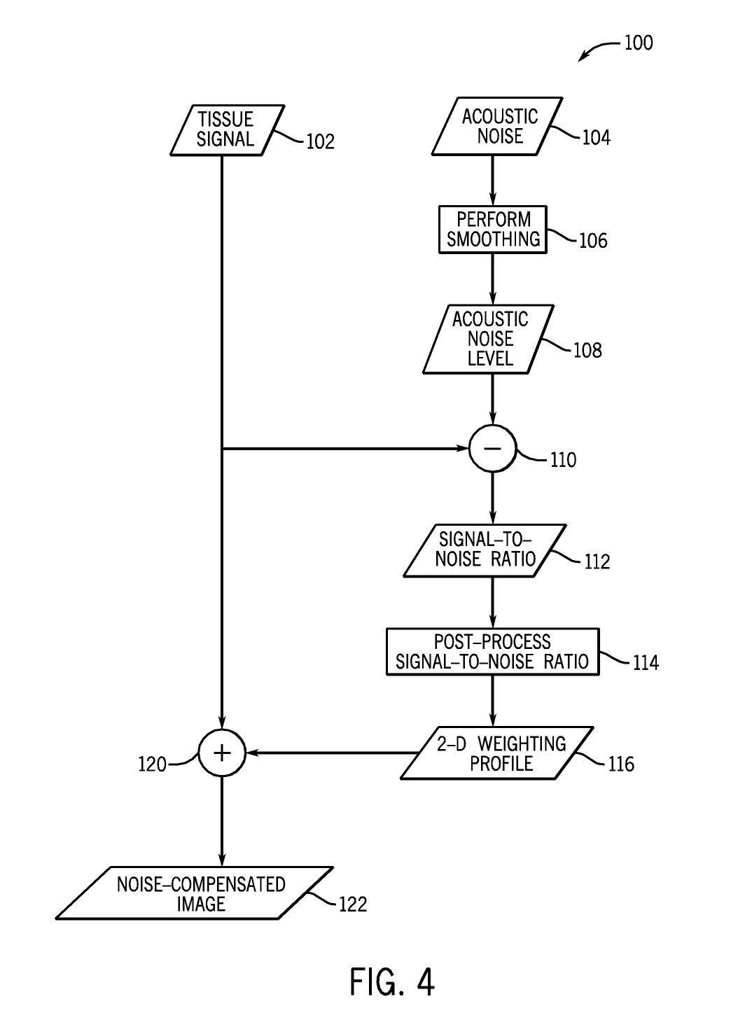

For example, with respect to image quality enhancement, an example on one approach for using acoustic noise measurements to improve image quality is depicted by FIG. 4. In particular, method 100 of FIG. 4 is depicted in flowchart form to describe various control logic and steps that may be implemented as an algorithm stored on a suitable medium and/or executed using one or more processing components of an ultrasound imaging system 10.

In this example, a tissue signal 102 and a measure of acoustic noise 104 are provided as inputs to the algorithm. In the depicted implementation, the measured acoustic noise 104 is smoothed (block 106) to obtain an acoustic noise level 108 (e.g., an average noise level). In one embodiment, the smoothing operation may include spatial averaging, such as over a 5×5 or 10×10 neighborhood or region. In such an embodiment, spatial averaging may be suitable where an average noise level is of interest and one is not concerned with structure.

In the depicted implementation, the acoustic noise level 108 is subtracted (block 110) from the tissue signal 102 to obtain a signal-to-noise ratio 112, as given by:

SNR(x)=t(x)−LPF(n(x)) (3)

where SNR is the signal-to-noise ratio 112, t is the tissue signal, n is the acoustic noise, and LPF(n) is the low pass filtered or otherwise smoothed noise. In one embodiment the derived signal-to-noise ratio may be approximately 10 dB. The signal-to-noise ratio 112 may be processed (block 114) to generate a two-dimensional weighting profile 116, as given by:

w(x)=f(SNR(x),n(x)). (4)

For example, in one embodiment, the signal-to-noise ratio 112 may be non-linearly processed to generate the two-dimensional weighting profile 116. In accordance with one implementation, the two-dimensional weighting profile 116 effectively suppresses areas with poor signal-to-noise and adjusts acoustic noise to a proper crossover level with an image generated based on the tissue signal 102. For example, in one embodiment, the two-dimensional weighting profile 116 has a corresponding weight for every coordinate (e.g. polar coordinates or Cartesian coordinates) in an image generated using the tissue signal, though in other embodiments not every coordinate may have a corresponding weight or each weight may correspond to more than one coordinate.

In the process (block 114) applied to the signal-to-noise ratio 112 to generate the two-dimensional weighting profile 116, the signal-to-noise ratio 112 may be thresholded, in one embodiment, to allow differential processing based on signal intensity or strength. That is, coordinates having a signal intensity of one strength may be processed differently (or not processed) than coordinates having a different signal intensity. In addition, all or part of the signal-to-noise ratio 112 may be scaled (i.e., multiplied by some fractional or whole number) to obtain a suitable intensity level (i.e., dB level) for the signal-to-noise ratio 112 as a whole or only for individual parts (i.e., coordinates) of the signal-to-noise ratio 112. Similarly, the signal-to-noise ratio 112 may under a smoothing operation (e.g., a spatial averaging operation) so that the tissue signal 102 contributions are smoothed.

Once obtained, the two-dimensional weighting profile 116 may be added (block 120) or otherwise applied (such as on a coordinate-by-coordinate basis) to the original tissue image to obtain a noise-compensated image 122 (e.g., a noise-reduced image), as given by:

tₒₚₜ(x)=t(x)+w(x) (5)

where tₒₚₜ is the optimized or noise-compensated tissue image 122. As will be appreciated, the enhancement algorithm may be applied to each image frame in real-time or near real-time. In one embodiment, the noise-compensated image 122 may have an improved contrast ratio when compared to the original tissue image, i.e. tissue signal 102.

Technical effects of the invention include operation of an ultrasound system such that receive beamforming occurs at angles offset from transmit beamforming to generate a measure of acoustic noise. Other effects include the execution on a processor of one or more algorithms that process acoustic noise measurements to generate a weighting profile that is combined with a tissue image to generate a noise-compensated or reduced tissue image.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An ultrasound system, comprising:
a probe comprising a plurality of transducer elements;
a station in communication with the probe, the station comprising:
transmit circuitry that controls the emission and direction of a transmitted ultrasound beam by the probe;
receive circuitry that controls the reception and direction of a received ultrasound beam by the probe, wherein the receive circuitry controls the direction of the received ultrasound beam to be the same as the transmitted beam when a tissue signal is being measured and controls the direction of the received ultrasound beam to be different than the transmitted beam when an acoustic noise signal is being measured; and
a controller directing operation of the transmit circuitry and the receive circuitry.

2. The ultrasound system of claim 1, wherein the tissue signal and the acoustic noise signal are separately measured

3. The ultrasound system of claim 1, wherein the tissue signal and the acoustic noise signal are measured simultaneously using multiple line acquisition.

4. The ultrasound system of claim 1, wherein beamforming of the transmitted ultrasound beam and of the received ultrasound beam is implemented via software executing on the controller.

5. The ultrasound system of claim 1, wherein a first delay profile associated with the received ultrasound beam is different than a second delay profile associated with the transmitted ultrasound beam when the acoustic noise signal is being measured.

6. The ultrasound system of claim 1, wherein the receive circuitry controls the direction of the received ultrasound beam to be offset from the direction of the transmitted beam by between about 15° to about 60° when an acoustic noise signal is being measured.

7. The ultrasound system of claim 1, wherein the received ultrasound beam is directed to a side lobe of an acoustic signal while the transmitted ultrasound beam is directed to a main lobe of the acoustic signal when acoustic noise is being measured.

8. A method for measuring acoustic noise associated with an ultrasound signal, the method comprising the acts of:
generating a transmit beam having a transmit steering direction determined at least in part by a transmit delay profile associated with the transmit beam;
generating a receive beam having a receive steering direction determined at least in part by a receive delay profile, wherein the receive delay profile associated with the receive beam results in substantially the same receive steering direction as the transmit steering direction when a tissue signal is being acquired and wherein the receive delay profile associated with the receive beam results in a receive steering direction that differs from the transmit steering direction when an acoustic noise signal is being measured.

9. The method of claim 8, wherein the receive delay profile causes the receive beam to be offset from the transmit beam by between about 15° to about 60° when an acoustic noise signal is being measured.

10. The method of claim 8, wherein the tissue signal and the acoustic noise signal are acquired substantially concurrently using multiple line acquisition.

11. The method of claim 8, comprising processing the tissue signal based on the acoustic noise signal to generate a noise-compensated tissue image.

12. The method of claim 8, wherein the receive beam and the transmit beam are focused in different directions when the receive delay profile is different from the transmit delay profile

13. The method of claim 8, comprising using the measured acoustic noise signal as an image quality indicator of probe contact or phase aberration.

14. A method for processing an ultrasound image, the method comprising the acts of:
acquiring a tissue signal and an acoustic noise signal;
deriving an acoustic noise level from the acoustic noise signal;
deriving a signal-to-noise ratio using the tissue signal and the acoustic noise signal;
processing the signal-to-noise ratio to generate a weighting profile;
applying the weighting profile to the tissue signal to generate a noise-compensated image.

15. The method of claim 14, wherein deriving the acoustic noise level from the acoustic noise signal comprises smoothing the acoustic noise signal.

16. The method of claim 14, wherein deriving the signal-to-noise ratio comprises subtracting the acoustic noise level from the tissue signal.

17. The method of claim 14, wherein the weighting profile corresponds to the tissue signal on a point-by-point basis.

18. The method of claim 14, wherein processing the signal-to-noise ratio comprises thresholding the signal-to-noise ratio based on signal intensity;

19. The method of claim 14, wherein processing the signal-to-noise ratio comprises scaling the signal-to-noise ratio to suitable decibel level.

20. The method of claim 14, wherein processing the signal-to-noise ratio comprises smoothing the signal-to-noise ratio.
